(19) United States
(12) Patent Application Publication
(10) Pub. No.: US 2023/0052826 A1
(43) Pub. Date: Feb. 16, 2023

(54) OIL-IN-WATER EMULSION SUNSCREEN COSMETIC

(71) Applicant: Shiseido Company, Ltd., Chuo-ku, Tokyo (JP)

(72) Inventors: Ayumu ENOMOTO, Tokyo (JP); Keita NISHIDA, Tokyo (JP)

(73) Assignee: Shiseido Company, Ltd., Chuo-ku, Tokyo (JP)

(21) Appl. No.: 17/788,573

(22) PCT Filed: Dec. 17, 2020
(86) PCT No.: PCT/JP2020/047176

§ 371 Date: Jun. 23, 2022

(30) Foreign Application Priority Data
Dec. 24, 2019 (JP) 2019-232819

(51) Int. Cl. A61K 8/81; A61K 8/06; A61K 8/25; A61Q 17/04

(52) U.S. Cl. CPC A61K 8/8147; A61K 8/062; A61K 8/8158; A61K 8/25; A61Q 17/04; A61K 2800/48; A61K 2800/621; A61K 2800/594

(57) ABSTRACT

An objective of the invention is to increase the ultraviolet protection power of an ultraviolet scattering agent, thereby providing an oil-in-water emulsion sunscreen cosmetic that can achieve high ultraviolet protection power and that has excellent emulsion stability. The sunscreen cosmetic according to the present invention comprises (A) an acrylic copolymer having phosphoric acid groups; (B) a hydrophobically treated ultraviolet scattering agent; and (C) 0.3% by mass or more of a water phase thickener; wherein the ultraviolet scattering agent is dispersed in an oil phase.

### TECHNICAL FIELD

The present invention relates to an oil-in-water emulsion sunscreen cosmetic. More specifically, the present invention relates to an oil-in-water emulsion sunscreen cosmetic that can achieve high ultraviolet protection power by blending a specific polymer compound in combination with an ultraviolet scattering agent.

### BACKGROUND ART

Oil-in-water emulsion cosmetics are widely used as bases in external preparations for use on the skin, such as skin cosmetics that are directly applied to the skin, for being able to obtain a fresh and watery feeling to the touch when applied to skin. In particular, protecting the skin from ultraviolet rays for skin care and body care has become something that is performed everyday, and the importance of using oil-in-water emulsion cosmetics as bases in such sunscreen cosmetics is increasing.

As ultraviolet protectants blended into sunscreen cosmetics, there are generally ultraviolet absorbing agents and ultraviolet scattering agents. Ultraviolet absorbing agents include those that are solid at ambient temperature, requiring a considerable amount of oil to be stably dissolved in cosmetics without being precipitated. For this reason, if high ultraviolet protection effects are to be obtained by blending a large amount of ultraviolet absorbing agents, then the amount of oils must also be increased, and the oils can cause stickiness to occur, degrading the feeling in use. Additionally, if high ultraviolet protection effects are to be obtained by increasing the blended amount of ultraviolet scattering agents, then the fluidity within the coating film is lowered, sometimes causing the feeling in use to become worse, such as having a feeling of powder squeakiness over time (a feeling to the touch in which the smoothness is gradually lost).

Therefore, technology for obtaining high ultraviolet protection effects with a lower blended amount of ultraviolet protectants has been proposed. For example, the applicant described, in Patent Document 1, that the ultraviolet protection power was increased by blending a porous spherical powder, such as silica, into an aqueous or oil-in-water sunscreen cosmetic in which an ultraviolet absorbing agent has been blended.

Regarding ultraviolet scattering agents as well, systems in which high ultraviolet protection effects can be obtained with lower blended amounts are sought. Additionally, in an oil-in-water sunscreen cosmetic, there is also difficulty in stably blending the ultraviolet protectant into the oil phase, which is the internal phase.

### RELATED ART

### Patent Documents

Patent Document 1: JP 2020-090468 A

### SUMMARY OF THE INVENTION

### Problem to be Solved by the Invention

An objective of the present invention is to increase the ultraviolet protection power of an ultraviolet scattering agent, thereby providing an oil-in-water emulsion sunscreen cosmetic that can achieve high ultraviolet protection power and that has excellent emulsion stability.

### Means for Solving the Problem

The present inventors performed diligent research towards solving the above-mentioned problem, as a result of which they discovered that the ultraviolet protection power of ultraviolet scattering agents increases when an acrylic copolymer having phosphoric acid groups, a hydrophobically treated ultraviolet scattering agent and a water phase thickener are combined and blended, thus completing the present invention.

That is, the present invention provides an oil-in-water emulsion sunscreen cosmetic comprising:

(A) an acrylic copolymer having phosphoric acid groups;
(B) a hydrophobically treated ultraviolet scattering agent; and
(C) 0.3% by mass or more of a water phase thickener;
wherein the ultraviolet scattering agent is dispersed in an oil phase.

### Effects of the Invention

By having the above-mentioned features, the cosmetic of the present invention can increase the ultraviolet protection power of the ultraviolet scattering agent blended into the sunscreen cosmetic and can realize an oil-in-water emulsion sunscreen cosmetic having excellent emulsion stability. Additionally, in the emulsion cosmetic of the present invention, the emulsion stability is sufficiently maintained, and thus, an ultraviolet absorbing agent that is a high-polarity oil can be further blended.

### MODES FOR CARRYING OUT THE INVENTION

The sunscreen cosmetic of the present invention is characterized by comprising (A) an acrylic copolymer having phosphoric acid groups, (B) a hydrophobically treated ultraviolet scattering agent, and (C) a water phase thickener. Hereinafter, the components constituting the cosmetic of the present invention will be described in detail.

### <(A) Acrylic Copolymer Having Phosphoric Acid Groups>

The (A) acrylic copolymer having phosphoric acid groups (hereinafter sometimes referred to simply as “component (A)”) blended in the sunscreen cosmetic of the present invention refers to a copolymer comprising acrylic monomers and acrylic monomers having phosphoric acid groups.

The acrylic monomers are monomers consisting of acrylic acid, methacrylic acid or alkyl esters thereof. The alkyl groups bonded by ester bonds may be linear or branched, and the number of carbon atoms therein should be 1 to 30, preferably 1 to 20. Examples include, but are not limited to, monomers of (meth)acrylic acid, methyl (meth)acrylate, oxyethyl (meth)acrylate, 2-ethylhexyl (meth)acrylate and the like.

Acrylic monomers having phosphoric acid groups are monomers in which the carboxyl groups in acrylic acid or methacrylic acid are esterized by means of alkyl groups, the acrylic monomers having phosphoric acid groups on the alkyl groups. Specifically, examples include, but are not limited to, phosphoxyethyl acrylate, phosphoxyethyl methacrylate, phosphoxymethyl acrylate and the like.

Specific examples of acrylic copolymers having phosphoric acid groups include, but are not limited to, “acrylates/methacryloyloxyethyl phosphate) copolymers.

The acrylic copolymer of the present invention may be provided as an aqueous dispersion in which acrylic polymer particles are dispersed in an aqueous solvent. As commercially available products, SOLTEX™ INO polymer (aqueous dispersion with 31% solids, manufactured by Dow Chemical Japan) and the like may be used.

The blended amount of the (A) acrylic copolymer having phosphoric acid groups should preferably be 0.1% to 10% by mass, more preferably 0.2% to 8% by mass and even more preferably 0.5% to 5% by mass in terms of solid content relative to the total amount of the sunscreen cosmetic. If the blended amount of the component (A) is less than 0.1% by mass, then the ultraviolet protection power increase effects will not be sufficient, and if more than 10% by mass is blended, then the emulsion stability tends to become worse.

### <(B) Hydrophobically Treated Ultraviolet Scattering Agent>

The (B) hydrophobically treated ultraviolet scattering agent (hereinafter sometimes referred to simply as “component (B)”) blended into the sunscreen cosmetic of the present invention is a powder that physically blocks ultraviolet rays by reflection or scattering, wherein the surface of the powder is hydrophobically treated.

The ultraviolet scattering agent of the present invention is not particularly limited, but may be a fine-particle metal oxide such as, for example, zinc oxide, titanium oxide, iron oxide, cerium oxide, tungsten oxide or the like. In the present invention, zinc oxide or titanium oxide is preferably used.

The average primary particle size of the ultraviolet scattering agent in the present invention is not particularly limited, but is preferably 10 nm to 100 nm or 10 nm to 50 nm. In this case, the average primary particle size in the present specification refers to the size of primary particles of the powder measured by a generally used method, specifically a value determined as the arithmetic mean of the lengths of the major axes of the particles and the lengths of the minor axes of the particles in transmission electron microscope images.

The shape of the ultraviolet scattering agent in the present invention is not particularly limited, but may be spherical, elliptical, crushed or the like.

The hydrophobic treatment agent in the ultraviolet scattering agent of the present invention may be various compounds that can be used for hydrophobic surface treatment of an ultraviolet scattering agent blended into cosmetics or the like, including, for example, fatty acids, silicone compounds, fluorine compounds, silane coupling agents, oils, quaternary ammonium salt compounds and the like. However, there are cases in which the desired ultraviolet protection power increase effects cannot be obtained when a fatty acid ester is used for hydrophobic treatment. Therefore, in the present invention, the (B) hydrophobically treated ultraviolet scattering agent refers to an “ultraviolet scattering agent hydrophobically surface treated with a compound other than a fatty acid ester”.

Examples of the fatty acid treatments include, but are not limited to, those using palmitic acid, isostearic acid, stearic acid, lauric acid, myristic acid, behenic acid, oleic acid, rosinic acid, 12-hydroxystearic acid and the like.

Examples of the silicone compounds include, but are not limited to, silicone oils such as methylhydrogen polysiloxane (hydrogen dimethicone), dimethyl polysiloxane (dimethicone) and methylphenyl polysiloxane.

Examples of the fluorine compounds include, but are not limited to, perfluoroalkyl group-containing esters, perfluropolyether, polymers having perfluoroalkyl groups and the like.

Examples of the silane coupling agents include, but are not limited to, fluoroalkylsilane compounds such as perfluoroalkylsilane, trifluoromethylethyl trimethoxysilane and heptadecafluorodecyl trimethoxysilane; and alkylsilane compounds such as methyl triethoxysilane, ethyl triethoxysilane, hexyl triethoxysilane and octyl triethoxysilane.

Examples of the oils include, but are not limited to, liquid paraffins, squalane, vaseline, lanolin, microcrystalline wax, polyethylene wax and the like.

Examples of the quaternary ammonium salt compounds include, but are not limited to, stearyl trimethyl ammonium chloride, behenyl trimethyl ammonium chloride, cetyl trimethyl ammonium chloride, distearyl dimethyl ammonium chloride, dibehenyl dimethyl ammonium chloride, dicetyl dimethyl ammonium chloride, stearyl dimethyl benzyl ammonium chloride, dilauryl dimethyl ammonium chloride and the like.

These hydrophobic treatments can be performed in accordance with conventional methods, and the hydrophobic treatment agents may be used as a single type or as a combination of two or more types.

The hydrophobic treatment of the ultraviolet scattering agent in the present invention is preferably a hydrophobic treatment using a silicone compound or a quaternary ammonium salt compound.

The ultraviolet scattering agent of the present invention may be coated with silica or alumina (aluminum oxide), and a silica- or alumina-coated ultraviolet scattering agent that is surface-treated by a hydrophobic surface treatment agent mentioned above may be used.

Examples of commercially available products that can be used as the hydrophobically surface-treated ultraviolet scattering agent in the present invention include, but are not limited to, OTQ-MT-100Si (distearyl dimethyl ammonium chloride-treated silica fine-particle titanium oxide, manufactured by Tayca Corp.), STR-100C-LP (hydrogen dimethicone/aluminum hydroxide-treated titanium oxide, manufactured by Sakai Chemical Industry Co., Ltd.), FINEX-50W-LP2 (hydrogen dimethicone/silica-treated zinc oxide, manufactured by Sakai Chemical Industry Co., Ltd.) and the like.

The blended amount of the (B) hydrophobically treated ultraviolet scattering agent is not particularly limited, but should normally be 1% by mass or higher, for example, 1% to 40% by mass, and preferably 1% to 30% by mass relative to the total amount of the cosmetic. If the blended amount of the (B) hydrophobically treated ultraviolet scattering agent is less than 1% by mass, then sufficient ultraviolet protection effects are difficult to obtain, and even if more than 40% by mass is blended, an increase in the ultraviolet protection effects that is commensurate with the blended amount cannot be expected, and the stability becomes worse.

### <(C) Water Phase Thickener>

As the (C) water phase thickener (hereinafter sometimes referred to simply as “component (C)”) blended in the sunscreen cosmetic of the present invention, one that is normally blended into cosmetics in order to increase the thickness of the water phase may be used. Specific examples include, but are not limited to, various types of hydrophilic thickeners such as natural water-soluble polymers, semi-synthetic water-soluble polymers, synthetic water-soluble polymers, inorganic thickeners and the like.

The natural water-soluble polymers include, for example, vegetable polymers such as gum arabic, tragacanth gum, galactan, guar gum, carrageenan, pectin, quinceseed (marmelo) extract, agar and brown algae powder; microbial polymers such as xanthan gum, dextran, pullulan and succinoglycan; and animal polymers such as collagen, casein, albumin and gelatin.

The semi-synthetic water-soluble polymers include, for example, starch-based polymers such as carboxymethyl starch and methylhydroxy starch; cellulose-based polymers such as methyl cellulose, nitrocellulose, ethyl cellulose, methyl hydroxypropyl cellulose, hydroxyethyl cellulose, stearoxyhydroxypropylmethyl cellulose, cellulose sulfuric acid salts, hydroxypropyl cellulose, carboxymethyl cellulose and crystalline cellulose; and alginic acid-based polymers such as sodium alginate, alginic acid propylene glycol esters and the like.

The synthetic water-soluble polymers include, for example, vinyl polymers such as polyvinyl alcohol, polyvinyl acetate, polyvinyl methyl ether, polyvinyl pyrrolidone, vinyl pyrrolidone/vinyl acetate copolymer and carboxyvinyl polymer; and acrylic polymers such as sodium polyacrylate, polyethyl acrylate, polyacrylamide, acrylic acid/alkyl methacrylate copolymers (for example, (acrylates/(C10-30) alkyl acrylate)) crosspolymer), alkanolamine polyacrylate, alkyl methacrylate/dimethylaminoethyl methacrylate copolymer, poly-2-acrylamido-2-methylpropane sulfonic acid, polymethacryloyloxy trimethyl ammonium, (ammonium acryloyldimethyl taurate/VP) copolymer, (dimethylacrylamide/sodium acryloyldimethyl taurate) copolymer and the like.

The inorganic thickeners include, for example, inorganic thickeners such as bentonite, laponite, hectorite, aluminum magnesium silicate, silicic anhydride and the like.

The (C) water phase thickener may be used as a single type or as a combination of two or more types.

The sunscreen cosmetic of the present invention tends to become sticky when a sugar-derived thickener is used. Thus, a water phase thickener other than a sugar-derived thickener is preferably used.

In particular, in the cosmetic of the present invention, one or more types selected from among acrylic acid/alkyl methacrylate copolymer, carboxyvinyl polymer, agar, stearoxyhydroxypropylmethyl cellulose, (dimethylacrylamide/sodium acryloyldimethyl taurate) copolymer and succinoglycan are preferably used.

The blended amount of the (C) water phase thickener should be 0.3% by mass or higher relative to the total amount of the cosmetic. Although the upper limit of the blended amount may be the usual upper limit value of the amount blended into cosmetics, it should preferably be, for example, 2% by mass or less. If the blended amount is less than 0.3% by mass, then the emulsion stability becomes worse.

By using a combination of two or more types selected from among the aforementioned water phase thickeners as component (C) in the sunscreen cosmetic of the present invention, a cosmetic with a further improved feeling to the touch in use can be obtained. For example, component (C) in the present invention is preferably a mixture of two or more types selected from among acrylic acid/alkyl methacrylate copolymer, carboxyvinyl polymer, agar, xanthan gum, stearoxyhydroxypropylmethyl cellulose, (dimethylacrylamide/sodium acryloyldimethyl taurate) copolymer and succinoglycan.

Additionally, although it is possible to use a sugar-derived thickener in the sunscreen cosmetic of the present invention, in the case in which a sugar-derived thickener (for example, xanthan gum, agar, succinoglycan or stearoxyhydroxypropylmethyl cellulose) is used, then it should preferably be combined with a water phase thickener having low salt tolerance in order to improve the feeling to the touch in use. Water phase thickeners having low salt tolerance are thickeners in which the viscosity becomes lower due to the presence of electrolytes at a concentration within a range in which they are generally blended into cosmetics, and they are also referred to as thickeners in which the viscosity is lowered by an increase in the electrolyte concentration.

The thickeners in which the viscosity is lowered by an increase in the electrolyte concentration are not particularly limited, but include, for example, vinyl polymers such as polyvinyl alcohol, polyvinyl acetate, polyvinyl methyl ether, polyvinyl pyrrolidone, vinyl pyrrolidone/vinyl acetate copolymer and carboxyvinyl polymer; and acrylic polymers such as sodium polyacrylate, polyethyl acrylate, alkanolamine polyacrylate, alkyl methacrylate/dimethylaminoethyl methacrylate copolymers, poly-2-acrylamido-2-methylpropane sulfonic acid, polymethacryloyloxy trimethyl ammonium, (ammonium acryloyldimethyl taurate/VP) copolymer, (dimethylacrylamide/sodium acryloyldimethyl taurate) copolymer and the like.

By formulating the sunscreen cosmetic of the present invention as an oil-in-water emulsion in which the ultraviolet scattering agent is dispersed in the oil phase, which is the internal phase, the ultraviolet protection power of the ultraviolet scattering agent can be improved.

The sunscreen cosmetic of the present invention preferably contains the (A) acrylic copolymer having phosphoric acid groups and the (C) water phase thickener at a blended mass ratio of 1:7 to 8:1 for the purposes of obtaining a cosmetic having excellent emulsion stability.

Since the sunscreen cosmetic of the present invention has excellent emulsion stability, a (D) ultraviolet absorbing agent (hereinafter sometimes referred to simply as “component (D)”), which is a high-polarity oil, may be further blended therein.

The ultraviolet absorbing agent used in the present invention is not particularly limited, but specific examples include organic ultraviolet absorbing agents such as ethylhexyl methoxycinnamate, octocrylene, dimethicodiethyl benzalmalonate, polysilicone-15, t-butylmethoxy dibenzoyl methane, ethylhexyl triazone, diethylamino hydroxybenzoyl hexyl benzoate, bis-ethylhexyloxyphenol methoxyphenyl triazine, oxybenzone-3, methylene bis-benzotriazolyl tetramethylbutyl phenol, phenylbenzimidazole sulfonic acid, homosalate and ethylhexyl salicylate. The ultraviolet absorbing agent used in the present invention may be blended as a single type or as a combination of two or more types.

In the case in which the (D) ultraviolet absorbing agent is blended, the amount thereof should preferably be 1% to 40% by mass, and more preferably 1% to 30% by mass relative to the total amount of the cosmetic.

By blending a (E) porous spherical powder having an average particle size of 1 to 4 μm and an oil absorption rate of 160 ml/100 g or less (hereinafter sometimes referred to simply as “component (E)”) into the sunscreen cosmetic of the present invention, the effect of further raising the ultraviolet protection power increase rate can be achieved.

The average particle size of the porous spherical powder blended as component (E) in the present invention is 1 to 4 μm and should more preferably be 1.5 to 4 μm. If the average particle size is smaller than 1 μm, then the cosmetic tends to become difficult to handle, and if the average particle size is larger than 4 μm, then high ultraviolet protection power increase effects cannot be obtained and there is a tendency for graininess to occur and for the texture to become worse. The average particle size in the present invention is obtained by adding 0.05 g of the powder to 20 g of an ethanol solvent, then subjecting the mixture to one minute of ultrasonic dispersion using an ultrasonic homogenizer (US-150T, manufactured by Nissei Corp.), and measuring the volume-average particle size (D50) by using a laser diffraction/scattering particle size distribution measurement device (MT3300EX II; manufactured by MicrotracBEL Corp.).

The oil absorption rate of the (E) porous spherical powder is the oil absorption rate measured in accordance with the JIS K5101-13-2 (boiled linseed oil method) standard, having a value of 160 ml/100 g or less, more preferably 50 to 160 ml/100 g, and even more preferably 60 to 160 ml/100 g. As long as the oil absorption rate is within the above-mentioned range, the ultraviolet protection effects can be sufficiently improved.

The (E) porous spherical powder has a shape that is spherical, particularly perfectly spherical. The closer the shape of component (E) is to perfectly spherical, the better a coating film of the cosmetic can be formed with a uniform thickness on the skin, thereby allowing high ultraviolet protection power increase effects to be achieved. In the present invention, perfectly spherical refers to being substantially perfectly spherical when viewed in projection from any direction, such that the smallest value of the particle size is 80% of more of the largest value, and more preferably 90% or more.

The material constituting the (E) porous spherical powder is not particularly limited, and may be silica (silicic anhydride), cellulose or the like, but silica is particularly preferable.

Commercial products that are silica powders that can be used as the (E) porous spherical powder include, for example, Godd Ball E-6C, Godd Ball B-6C (the above manufactured by Suzukiyushi Industrial Corp.), Silica Microbead P-500 (manufactured by JGC C&C Ltd.), Sunsphere L-31 (manufactured by AGC Si-Tech Co., Ltd.) and the like.

The (E) porous spherical powder may be used as a single type alone, or as a combination of two or more types. The blended amount of component (E) should preferably be 1% to 5% by mass and more preferably 2% to 4% by mass relative to the total amount of the sunscreen cosmetic. If the blended amount of component (E) is less than 1% by mass, then the ultraviolet protection power increase effect due to component (E) tends to be insufficiently achieved, and if more than 5% by mass is blended, then there are cases in which the texture becomes worse.

### <Optional Blended Components>

Aside from the above-mentioned components, components that are normally used in cosmetics may be blended into the sunscreen cosmetic of the present invention within a range not compromising the effects of the present invention. For example, oils, water, alcohols, humectants, oil phase thickeners, surfactants, film formation agents, powder components, medicinal components, stabilizers, chelating agents, preservatives, fragrances and the like may be appropriately blended as needed.

The sunscreen cosmetic of the present invention can be produced by a conventional method. For example, a liquid mixture obtained by heating and mixing the oil phase components may be added to a liquid mixture of the water phase components that have been separately heated and mixed, an aqueous dispersion of the acrylic copolymer having phosphoric acid groups may be added to the resulting liquid mixture, and the liquid mixture may be stirred well to produce the oil-in-water emulsion cosmetic.

The sunscreen cosmetic of the present invention may be prepared as a sunscreen cream, a sunscreen milky lotion or a sunscreen lotion, as well as a toner or the like provided with sunscreen effects.

The present invention is based on the discovery that the ultraviolet protection power of the ultraviolet scattering agent is particularly significantly increased by combining an acrylic copolymer, which has the function of preventing aggregation of the ultraviolet scattering agent in a cosmetic coating film on the skin, with a specific blended amount of a water phase thickener to produce an oil-in-water emulsion. The ultraviolet protection power increase mechanism of the present invention differs from the conventional ultraviolet protection power increase mechanism that is based on making the cosmetic coating film uniform. Thus, even when combined with porous spherical powders or the like, which provide the conventional mechanism, the mechanisms will not conflict with each other, and additive or synergistic ultraviolet protection power increase effects can be obtained.

In the sunscreen cosmetic of the present invention, the ultraviolet protection power can be sufficiently increased even without blending hollow polymers (for example, a hollow polymer that is a (styrene/acrylates) copolymer), which have been reported as providing conventional ultraviolet protection increase effects. Thus, the embodiments of the present invention include embodiments not comprising hollow polymers.

### Examples

Although the present invention will be explained in further detail by providing examples below, the present invention is not limited in any way thereby. Where not otherwise noted, the blended amounts are indicated in percentage by mass relative to the total amount of the system in which the relevant component is blended. Before specifically explaining each example, the evaluation methods that were employed will be explained.

### <Ultraviolet Protection Power Increase Rate>

Cosmetics (samples) according to each example were dripped, at a rate of 2 mg/cm², onto measurement plates (S plates) (5×5 cm V-groove PMMA (polymethyl methacrylate) plates, SPFMASTER-PA01), applied by finger for 60 seconds and dried for 15 minutes to form coating films, the absorbances of which were measured using a Hitachi U-3500 self-recording spectrophotometer. The absorbances (Abs) were computed, with an uncoated plate as the control, by using the following equation, and the measurement values were integrated from 280 nm to 400 nm to determine the absorbance integral values.

Abs=−log (T/To)

T: transmittance of sample, To: transmittance of uncoated plate

From the absorbance integral values of the samples that were determined, the ultraviolet protection power increase rates, relative to a control sample, were computed by the following equation. For Test Examples 1 to 7, samples of each test example in which component (A) was not blended were used as the control samples. For Test Examples 10 to 12, samples in which component (A) of the present invention and silica were not blended were used as the control samples.

[Ultraviolet protection power increase rate (%)]=[Absorbance integral value of sample]/[Absorbance integral value of control sample]×100

### <Emulsion Stability: Dispersibility of Hydrophobically Treated Ultraviolet Scattering Agent>

Evaluations were visually performed by observing the samples in the states after formulation, and by observing the states two weeks after screw tubes were filled with the samples.

### <Evaluation Criteria>

A: Stable emulsification was achieved, and separation or aggregation over time was not observed.
B: Stable emulsification was achieved, and some separation or aggregation over time was observed but there were no problems in use.
C: Stable emulsification was achieved, but separation or aggregation over time was observed.
D: Emulsification was not achieved.

### 1. Impact of Format on Ultraviolet Protection Power Increase Effects

Oil-in-water emulsion cosmetics (Test Example 1 and Test Example 2) and a water-in-oil emulsion cosmetic (Test Example 3) having the compositions indicated in Table 1 below were formulated by means of conventional methods. The ultraviolet protection power increase effects were evaluated in accordance with the above-described evaluation method.

TABLE 1

| | O/W emulsion cosmetic | | | | W/O emulsion cosmetic | |
|---|---|---|---|---|---|---|
| | | Test Ex 1 | | Test Ex 2 | | Test Ex 3 |
| Component name | Test Ex 1 | Control | Test Ex 2 | Control | Test Ex 3 | Control |
| Water | bal | bal | bal | bal | bal | bal |
| Ethanol | 8 | 8 | 10 | 10 | 10 | 10 |
| Phenoxyethanol | 0.5 | 0.5 | 0.5 | 0.5 | — | — |
| Glycerin | 1 | 1 | 4 | 4 | 1 | 1 |
| PEG-6 | — | — | 8 | 8 | — | — |
| Dimethyl distearyl ammonium hectorite | — | — | — | — | 0.5 | 0.5 |
| Dextrin palmitate | 1 | 1 | — | — | 1 | 1 |
| (Acrylates/(C10-30) alkyl acrylate) | 0.1 | 0.1 | — | — | — | — |
| crosspolymer | | | | | | |
| Carbomer | 0.15 | 0.15 | — | — | — | — |
| Agar | 0.1 | 0.1 | — | — | — | — |
| Xanthan gum | 0.1 | 0.1 | — | — | — | — |
| Stearoxyhydroxypropylmethyl celluose | — | — | 0.2 | 0.2 | — | — |
| (Dimethylacrylamide/sodium | — | — | 0.3 | 0.3 | — | — |
| acryloyldimethyl taurate) copolymer | | | | | | |
| PEG-9 polydimethyl polysiloxyethyl | — | — | — | — | 1.5 | 1.5 |
| dimethicone | | | | | | |
| (Acrylates/methoxy PEG-90 | 10 | 10 | — | — | — | — |
| methacrylate) crosspolymer (core-corona | | | | | | |
| microgel emulsifier) | | | | | | |
| Lauryl dimethyl aminoacetic acid betaine | 0.2 | 0.2 | — | — | — | — |
| 35% aqueous solution | | | | | | |
| Trimethylsiloxysilicic acid | — | — | — | — | 3 | 3 |
| Isododecane | — | — | — | — | 3 | 3 |
| Dimethicone (1.5 cst) | 8 | 8 | — | — | 20 | 20 |
| Isostearic acid | — | — | — | — | 0.5 | 0.5 |
| Diisopropyl sebacate | 7 | 7 | 10 | 10 | 6 | 6 |
| Sorbitan sesquiisostearate | — | — | 0.5 | 0.5 | — | — |
| Octocrylene | — | — | — | — | 3 | 3 |
| Ethylhexyl methoxycinnamate | 7.5 | 7.5 | 3 | 3 | 7.5 | 7.5 |
| bis-Ethylhexyloxyphenol methoxyphenyl | 3 | 3 | 3 | 3 | 0.5 | 0.5 |
| triazine | | | | | | |
| Ethylhexyl triazine | 3 | 3 | 3 | 3 | — | — |
| Diethylamino hydroxybenzoyl hexyl | 2 | 2 | — | — | 1.5 | 1.5 |
| benzoate | | | | | | |
| Oxybenzone-3 | 1 | 1 | 1 | 1 | — | — |
| Stearic acid/Al hydroxide-treated fine- | — | — | — | — | 2 | 2 |
| particle titanium oxide | | | | | | |
| Distearyl dimethyl ammonium chloride- | 2 | 2 | — | — | — | — |
| treated silica fine-particle titanium oxide | | | | | | |
| Hydrogen dimethicone/Al hydroxide- | — | — | 4 | 4 | — | — |
| treated titanium oxide | | | | | | |
| Hydrogen dimethicone/dimethicone- | — | — | — | — | 5 | 5 |
| treated fine-particle zinc oxide | | | | | | |
| Distearyl dimethyl ammonium chloride- | — | — | — | — | 6 | 6 |
| treated fine-particle zinc oxide | | | | | | |
| Dimethicone-treated talc | — | — | — | — | 5 | 5 |
| Methyl methacrylate crosspolymer | 2 | 2 | — | — | 7 | 7 |
| EDTA•3Na—2H2O | — | — | — | — | 0.1 | 0.1 |
| Potassium hydroxide | 0.15 | 0.15 | — | — | — | — |
| Tranexamic acid | — | — | 2 | 2 | — | — |
| (Acrylates/methacryloyloxyethyl | 1.55 | — | 1.55 | — | 1.55 | — |
| phosphate) copolymer | | | | | | |
| Total | 100 | 100 | 100 | 100 | 100 | 100 |
| Evaluation: Ultraviolet protection power | 122.40% | — | 121.60% | — | 97.1% | — |
| increase rate (%) when using each control | | | | | | |
| sample as reference | | | | | | |

As shown in Table 1, Test Example 1 and Test Example 2, which were formulated as oil-in-water emulsions, both had significantly increased ultraviolet protection power in comparison with control samples in which component (A) was not blended.

Meanwhile, an increase in the ultraviolet protection power was not observed in Test Example 3, which was formulated as a water-in-oil emulsion.

### 2. Impact of Hydrophobic Treatment Agent of Ultraviolet Scattering Agent on Ultraviolet Protection Power Increase Effects

Oil-in-water emulsion cosmetics having the compositions indicated in Table 2 below were formulated by means of conventional methods. The ultraviolet protection power increase effects were evaluated in accordance with the above-described evaluation method.

TABLE 2

| | | Test Ex 4 | | Test Ex 5 | | Test Ex 6 | | Test Ex 7 |
|---|---|---|---|---|---|---|---|---|
| Component name | Test Ex 4 | Control | Test Ex 5 | Control | Test Ex 6 | Control | Test Ex 7 | Control |
| Water | bal | bal | bal | bal | bal | bal | bal | bal |
| Ethanol | 5 | 5 | 5 | 5 | 5 | 5 | 5 | 5 |
| Glycerin | 3 | 3 | 3 | 3 | 3 | 3 | 3 | 3 |
| 1,3-Butylene glycol | [symbol] | [symbol] | [symbol] | [symbol] | [symbol] | [symbol] | [symbol] | [symbol] |
| Polyoxyethylene (14) | 2 | 2 | 2 | 2 | 2 | 2 | 2 | 2 |
| polyoxypropylene (7) dimethyl ether | | | | | | | | |
| ( [symbol] /sodium | 0.3 | 0.3 | 0.3 | 0.3 | 0.3 | 0.3 | 0.3 | 0.3 |
| acryloyldimethyl taurate) copolymer | | | | | | | | |
| [symbol] glycan | 0.12 | 0.12 | 0.12 | 0.12 | 0.12 | 0.12 | 0.12 | 0.12 |
| PEG-100 hydrogenated castor oil | 0.2 | 0.2 | 0.2 | 0.2 | 0.2 | 0.2 | 0.2 | 0.2 |
| Polyoxyethylene hardened castor oil (60 mol) | 1.3 | 1.3 | 1.3 | 1.3 | 1.3 | 1.3 | 1.3 | 1.3 |
| Dimethicone (1.5 cst) | 10 | 10 | 10 | 10 | 10 | 10 | 10 | 10 |
| Cyclopentasiloxane | 10 | 10 | 10 | 10 | 10 | 10 | 10 | 10 |
| Isostearic acid | 1 | 1 | 1 | 1 | 1 | 1 | 1 | 1 |
| Ethylhexyl methoxycinnamate | 7 | 7 | 7 | 7 | 7 | 7 | 7 | 7 |
| bis-Ethylhexyloxyphenol | 2 | 2 | 2 | 2 | 2 | 2 | 2 | 2 |
| methoxyphenyl triazine | | | | | | | | |
| Ethylhexyl triazine | 1 | 1 | 1 | 1 | 1 | 1 | 1 | 1 |
| Distearyl dimethyl ammonium | 15 | 15 | — | — | — | — | — | — |
| chloride-treated fine-particle | | | | | | | | |
| zinc oxide | | | | | | | | |
| Silica/dimethicone-treated | — | — | 15 | 15 | — | — | — | — |
| fine-particle zinc oxide | | | | | | | | |
| Hydrogen dimethicone-treated | — | — | — | — | 15 | 15 | — | — |
| fine-particle zinc oxide | | | | | | | | |
| Dextrin palmitate-treated | — | — | — | — | — | — | 15 | 15 |
| fine-particle zinc oxide | | | | | | | | |
| Spherical silica | 1.5 | 1.5 | 1.5 | 1.5 | 1.5 | 1.5 | 1.5 | 1.5 |
| Tranexamic acid | 2 | 2 | 2 | 2 | 2 | 2 | 2 | 2 |
| (Acrylates/methacryloyloxyethyl | 1.55 | — | 1.55 | — | 1.55 | — | 1.55 | — |
| phosphate) copolymer | | | | | | | | |
| Total | 100 | 100 | 100 | 100 | 100 | 100 | 100 | 100 |
| Evaluation: Ultraviolet protection power | 117% | — | 112.9% | — | 1 [symbol] % | — | 87% | — |
| increase rate (%) when using each control | | | | | | | | |
| sample as reference | | | | | | | | |

[symbol]

indicates data missing or illegible when filed

Spherical silica in table: Sunsphere L-51S (average particle size 5 μm, manufactured by AGC Si-Tech Co., Ltd.)

As shown in Table 2, Test Example 4, in which a distearyl dimethyl ammonium chloride-treated ultraviolet scattering agent was blended, Test Example 5, in which a dimethicone-treated ultraviolet scattering agent was blended, and Test Example 6, in which a hydrogen dimethicone-treated ultraviolet scattering agent was blended, had significantly increased ultraviolet protection power in comparison with control samples in which component (A) was not blended.

Meanwhile, an increase in the ultraviolet protection power was not observed in Test Example 7, in which a dextrin palmitate-treated ultraviolet scattering agent was blended.

### 3. Impact of Blended Amount of Water Phase Thickener

Oil-in-water emulsion cosmetics having the compositions indicated in Table 3 below were prepared by means of conventional methods. The emulsion stabilities were evaluated in accordance with the above-described evaluation method.

TABLE 3

| | Test | Text |
|---|---|---|
| Component name | Ex 8 | Ex 9 |
| Water | bal | bal |
| Ethanol | 5 | 5 |
| Glycerin | 3 | 3 |
| 1,3-Butylene glycol | 5 | 5 |
| Polyoxyethylene (14) polyoxypropylene | 2 | 2 |
| (7) dimethyl ether | | |
| (Dimethylacrylamide/sodium | 0.3 | 0.15 |
| acryloyldimethyl taurate) copolymer | | |
| Succinoglycan | 0.12 | 0.06 |
| PEG-100 hydrogenated castor oil | 0.2 | 0.2 |
| Polyoxyethylene hardened castor oil | 1.3 | 1.3 |
| (60 mol) | | |
| Dimethicone (1.5 cst) | 10 | 10 |
| Cyclopentasiloxane | 10 | 10 |
| Isostearic acid | 1 | 1 |
| Ethylhexyl methoxycinnamate | 7 | 7 |
| bis-Ethylhexyloxyphenol | 2 | 2 |
| methoxyphenyl triazine | | |
| Ethylhexyl triazine | 1 | 1 |
| Distearyl dimethyl ammonium chloride- | 15 | 15 |
| treated fine-particle zinc oxide | | |
| Spherical silica | 1.5 | 1.5 |
| Tranexamic acid | 2 | 2 |
| (Acrylates/methacryloyloxyethyl | 1.55 | 1.55 |
| phosphate) copolymer | | |
| Total | 100 | 100 |
| Evaluation: Emulsion stability | A | C |

Spherical silica in table: Sunsphere L-51S (average particle size 5 μm, manufactured by AGC Si-Tech Co., Ltd.)

As shown in Table 3, Test Example 8, in which the blended amount of the water phase thickener ((dimethylacrylamide/sodium acryloyl dimethyl taurate) copolymer and succinoglycan) was 0.3% by mass or higher, had excellent emulsion stability.

However, Test Example 9, in which the blended amount of the water phase thickener was less than 0.3% by mass, had poor emulsion stability.

### 4. Impact of Combined Usage of Porous Spherical Powder on Ultraviolet Protection Power Increase Effects

Oil-in-water emulsion cosmetics having the compositions indicated in Table 4 below were prepared by means of conventional methods. The ultraviolet protection power increase effects were evaluated in accordance with the above-described evaluation method.

TABLE 4

| | Test | Text | Test | Control |
|---|---|---|---|---|
| Component name | Ex 10 | Ex 11 | Ex 12 | Sample |
| Water | bal | bal | bal | bal |
| Ethanol | 8 | 8 | 8 | 8 |
| Phenoxyethanol | 0.5 | 0.5 | 0.5 | 0.5 |
| Glycerin | 1 | 1 | 1 | 1 |
| Dextrin palmitate | 1 | 1 | 1 | 1 |
| (Acrylates/(C10-30) alkyl | 0.1 | 0.1 | 0.1 | 0.1 |
| acrylate) crosspolymer | | | | |
| Carbomer | 0.15 | 0.15 | 0.15 | 0.15 |
| Agar | 0.1 | 0.1 | 0.1 | 0.1 |
| Xanthan gum | 0.1 | 0.1 | 0.1 | 0.1 |
| (Acrylates/methoxy PEG-90 | 10 | 10 | 10 | 10 |
| methacrylate) crosspolymer (core- | | | | |
| corona microgel emulsifier) | | | | |
| Lauryl dimethyl aminoacetic acid | 0.2 | 0.2 | 0.2 | 0.2 |
| betaine 35% aqueous solution | | | | |
| Dimethicone (1.5 cst) | 8 | 8 | 8 | 8 |
| Diisopropyl sebacate | 7 | 7 | 7 | 7 |
| Ethylhexyl methoxycinnamate | 7.5 | 7.5 | 7.5 | 7.5 |
| bis-Ethylhexyloxyphenol | 3 | 3 | 3 | 3 |
| methoxyphenyl triazine | | | | |
| Ethylhexyl triazine | 3 | 3 | 3 | 3 |
| Diethylamino hydroxybenzoyl | 2 | 2 | 2 | 2 |
| hexyl benzoate | | | | |
| Oxybenzone-3 | 1 | 1 | 1 | 1 |
| Distearyl dimethyl ammonium | 2 | 2 | 2 | 2 |
| chloride-treated silica fine- | | | | |
| particle titanium oxide | | | | |
| Methyl methacrylate crosspolymer | 2 | 2 | 2 | 2 |
| Silica | 4 | — | 4 | — |
| Potassium hydroxide | 0.15 | 0.15 | 0.15 | 0.15 |
| (Acrylates/methacryloyloxyethyl | 1.55 | 1.55 | — | — |
| phosphate) copolymer | | | | |
| Total | 100 | 100 | 100 | 100 |
| Evaluation: Ultraviolet | 129.9% | 122.4% | 105.9% | — |
| protection power increase | | | | |
| rate (%) when using control | | | | |
| sample as reference | | | | |

Silica in table: Godd Ball E-6C (Suzukiyushi Industrial Corp.)

Test Example 12 in Table 4 contains silica, which is mentioned as a component that provides ultraviolet protection power increase effects in Patent Document 1. Test Example 12, in which silica was blended, had improved ultraviolet protection power in comparison with a control sample in which silica was not blended. Additionally, Test Example 10, in which (acrylates/methacryloyloxyethyl phosphate) copolymer, which is component (A) in the present invention, was blended in combination with silica, was observed to have additive or synergistic ultraviolet protection power increase effects in comparison with Test Example 11 and Test Example 12, in which either one was blended alone.

1. An oil-in-water emulsion sunscreen cosmetic comprising:
(A) an acrylic copolymer having phosphoric acid groups;
(B) a hydrophobically treated ultraviolet scattering agent; and
(C) 0.3% by mass or more of a water phase thickener;
wherein the ultraviolet scattering agent is dispersed in an oil phase.

2. The cosmetic according to claim 1, wherein a blended mass ratio between component (A) and component (C) is 1:7 to 8:1.

3. The cosmetic according to claim 1, wherein component (B) is an ultraviolet scattering agent that is hydrophobically surface-treated by one or more compounds selected from among silicone compounds and quaternary ammonium salt compounds.

4. The cosmetic according to claim 1, further comprising (D) an ultraviolet absorbing agent.

5. The cosmetic according to claim 1,
wherein component (C) is a mixture of two or more compounds selected from among (acrylates/(C10-30) alkyl acrylate) crosspolymer, carboxyvinyl polymer, agar, stearoxyhydroxypropylmethyl cellulose, (dimethylacrylamide/sodium acryloyldimethyl taurate) copolymer and succinoglycan.

6. The cosmetic according to claim 1, further comprising (E) a porous spherical powder having an average particle size of 1 to 4 μm and having an oil absorption rate of 160 ml/100 g or less.
